(19) United States
(12) Patent Application Publication
(10) Pub. No.: US 2020/0368271 A1
(43) Pub. Date: Nov. 26, 2020

(54) METHOD AND APPARATUS FOR ADMINISTERING GASES INCLUDING NITRIC OXIDE

(71) Applicant: VERO Biotech LLC, Atlanta, GA (US)

(72) Inventors: Kurt A. DASSE, Needham, MA (US); Priscilla C. PETIT, Orlando, FL (US); David H. FINE, Cocoa Beach, FL (US); Mark K. WEDEL, Temecula, CA (US)

(21) Appl. No.: 16/773,707

(22) Filed: Jan. 27, 2020

Related U.S. Application Data
(63) Continuation of application No. 15/375,104, filed on Dec. 11, 2016, now abandoned.
(60) Provisional application No. 62/336,731, filed on May 15, 2016.
(60) Provisional application No. 62/266,466, filed on Dec. 11, 2015.

(51) Int. Cl. A61K 33/00; G01N 33/68; A61P 7/00; A61P 7/06; A61M 16/10; A61K 9/00; A61M 16/12

(52) U.S. Cl. CPC A61K 33/00; G01N 33/6887; A61P 7/00; A61P 7/06; A61M 16/104; A61K 9/0073; A61M 2016/0027; A61M 16/12; G01N 2333/78; A61M 2205/36; A61M 2205/3561; G01N 2800/7052; A61M 2016/1035; A61M 16/1005

(57) ABSTRACT

A method of modulating oxygen saturation levels can include measuring oxygen saturation levels in a patient, administering inhaled nitric oxide, adjusting the dose of oxygen in real time to a second dose based on the inhaled nitric oxide.

### PRIORITY CLAIM

This application is a continuation of U.S. patent application Ser. No. 15/375,104, filed Dec. 11, 2016, which claims priority under 35 U.S.C. § 119(e) to U.S. Provisional Application No. 62/266,466, filed Dec. 11, 2015 and U.S. Provisional Application No. 62/336,731, filed May 15, 2016, each of which are incorporated by reference in their entirety.

### TECHNICAL FIELD

The invention relates to mixing a gas flow including oxygen and a gas flow including a nitric oxide-releasing agent within a receptacle, which can be a cartridge, which converts the nitric oxide-releasing agent to nitric oxide.

### BACKGROUND

Understanding the effects of O₂ administration is important to prevent inadvertent alveolar damage caused by hyperoxia in patients requiring supplemental oxygenation.

Several pathophysiological processes are associated with increased levels of hyperoxia-induced reactive O₂ species (ROS) which may readily react with surrounding biological tissues, damaging lipids, proteins, and nucleic acids. Protective antioxidant defenses can become overwhelmed with ROS leading to oxidative stress. While all forms of aerobic life have evolved antioxidant defenses to cope with this potential problem, cellular antioxidants can become overwhelmed by oxidative insults, including supraphysiologic concentrations of O₂ (hyperoxia).

Nitric oxide, also known as nitrosyl radical, is a free radical that is an important signaling molecule. For example, NO can cause smooth muscles in blood vessels to relax, thereby resulting in vasodilation and increased blood flow through the blood vessel. These effects can be limited to small biological regions since NO can be highly reactive with a lifetime of a few seconds and can be quickly metabolized in the body.

Some disorders or physiological conditions can be mediated by inhalation of nitric oxide. The use of low concentrations of inhaled nitric oxide can prevent, reverse, or limit the progression of disorders which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism and idiopathic or primary pulmonary hypertension or chronic hypoxia.

Generally, nitric oxide can be inhaled or otherwise delivered to the individual's lungs. Providing a therapeutic dose of NO could treat a patient suffering from a disorder or physiological condition that can be mediated by inhalation of NO or supplement or minimize the need for traditional treatments in such disorders or physiological conditions. Typically, the NO gas can be supplied in a bottled gaseous form diluted in nitrogen gas (N₂). Great care should be taken to prevent the presence of even trace amounts of oxygen (O₂) in the tank of NO gas because the NO, in the presence of O₂, can be oxidized to nitrogen dioxide (NO₂). Unlike NO, the part per million levels of NO₂ gas can be highly toxic if inhaled and can form nitric and nitrous acid in the lungs.

### SUMMARY

A method of modulating oxygen saturation levels can include measuring oxygen saturation levels in a patient, administering inhaled nitric oxide, adjusting the dose of oxygen in real time to a second dose based on the inhaled nitric oxide, determining a first oxygen requirement to address an oxygen deficiency, determining a reduced oxygen requirement based on the generated nitric oxide, and delivering a dose of supplemental oxygen based on the reduced oxygen requirement and the gas mixture including nitric oxide from the receptacle to the patient.

The method can further include mixing a first gas including oxygen and a second gas including a nitric oxide-releasing agent within a receptacle to form a gas mixture, wherein the receptacle includes an inlet, an outlet and a reducing agent, and contacting the nitric oxide-releasing agent in the gas mixture with the reducing agent to generate nitric oxide.

In certain embodiments, adjusting the dose includes titrating the dose of oxygen in real time.

In other examples, a method of modulating oxygen saturation levels can include measuring oxygen saturation levels in a patient, determining a first dose of oxygen to address an oxygen deficiency, mixing a first gas including oxygen and a second gas including a nitric oxide, determining a second dose of oxygen based on an amount of nitric oxide to be co-administered with the oxygen, wherein the second dose is lower than the first dose, and delivering the gas mixture including nitric oxide from the receptacle to the patient.

The method of modulating oxygen saturation levels can also include an incremental reduction of pO2.

In certain embodiments, the method of modulating oxygen saturation levels is performed to reduce oxygen-induced inflammation.

This method can include reducing lung fibrosis. The method can also include reducing oxidative stress. The method can also be performed to address oxygen deficiency due to high altitude

In certain embodiments, the nitric oxide-releasing agent is nitrogen dioxide.

In certain embodiments, the method of modulating oxygen saturation levels, further includes delivering a hydrogen gas.

In certain embodiments, the second gas includes an inert gas or oxygen.

In other embodiments, the concentration of nitric oxide in the gas mixture delivered is at least 0.01 ppm and at most 2 ppm.

In yet other embodiments, the patient is treated for symptoms of interstitial lung disease, oxygen-induced inflammation, cardiac ischemia, myocardial dysfunction, ARDS, pneumonia, pulmonary embolism, COPD, emphysema, fibrosis, or mountain sickness due to high altitude.

In yet other embodiments, the nitric oxide is provided in an effective amount to minimize hemolysis during sepsis.

In certain embodiments, the hydrogen acts to eliminate peroxynitrite, thereby reducing adverse effects of nitric oxide.

In other embodiments, delivering the gas mixture including nitric oxide from the receptacle to the mammal includes passing the gas mixture through a delivery conduit located between the receptacle and a patient interface.

In some embodiments, the volume of the receptacle is greater than the volume of the delivery conduit.

In certain embodiments, the volume of the receptacle is at least two times the volume of the delivery conduit.

In certain embodiments, delivering the gas mixture including nitric oxide from the receptacle to the mammal includes intermittently providing the gas mixture to the mammal.

In other embodiments, delivering the gas mixture including nitric oxide from the receptacle to the mammal includes pulsing the gas mixture.

In some embodiments, pulsing includes providing the gas mixture for one or more pulses of 1 to 6 seconds.

In other embodiments, the volume of the receptacle is greater than the volume of the gas mixture in a pulse.

In yet other embodiments, the volume of the receptacle is at least twice the volume of the gas mixture in a pulse.

In some embodiments, the gas mixture is stored in the receptacle between pulses.

In other embodiments, the method of modulating oxygen saturation levels further includes storing the gas mixture in the receptacle for a predetermined period of time, and wherein the predetermined period is at least 1 second.

In some embodiments, pulsing includes providing the gas mixture for two or more pulses and the concentration of nitric oxide in each pulse varies by less than 10%.

In other embodiments, pulsing includes providing the gas mixture for two or more pulses and the concentration of nitric oxide in each pulse varies by less than 10 ppm.

In other embodiments, the method further includes communicating the first gas through a gas conduit to the receptacle and supplying the second gas into the gas conduit immediately prior to the receptacle.

In other examples, the method of modulating oxygen saturation levels includes supplying the second gas at the receptacle.

In yet other examples, the method of modulating oxygen saturation levels further includes administering exogenous NO in an amount effective to modulate the hormesis characteristics of NO.

In certain examples, the nitric oxide is administered to neonates.

In other embodiments, the nitric oxide is administered to pediatric patients.

In yet other embodiments, the nitric oxide is administered to adults.

In certain embodiments, the NO can be provided through a cartridge that converts nitric oxide-releasing agents to NO. The cartridge can include an inlet, an outlet, and a reducing agent. The cartridge can be configured to utilize the whole surface area in converting nitric oxide-releasing agents to NO. The cartridge can have a length, width, and thickness, an outer surface, and an inner surface, and can be substantially cylindrical in shape. The cartridge can have aspect ratio of approximately 2:1, 3:1 or 4:1. The length can be, for example, one inch, two inches, three inches, four inches or five inches. The width can be, for example, 0.5 inch, 1 inch, 1.5 inches, 2 inches, or 2.5 inches. The cartridge can have a cross-section that is a circle, oval, or ellipse. In certain embodiments, opposing sides along the length of the cartridge can be flat. The thickness between the inner and outer surface can be constant, thereby providing a uniform exposure to the reducing agents. The thickness can be approximately 1 mm, 2 mm, 5 mm, 10 mm, 20 mm, 30 mm, or 40 mm for example.

In certain embodiments, a method of modulating oxygen saturation levels, includes implanting a pulmonary artery pressure sensor, monitoring pulmonary artery pressure in real time, measuring oxygen saturation levels in a patient, administering inhaled nitric oxide, adjusting the dose of oxygen in real time to a second dose based on the inhaled nitric oxide, determining a first oxygen requirement to address an oxygen deficiency, determining a reduced oxygen requirement based on the generated nitric oxide, and delivering a dose of supplemental oxygen based on the reduced oxygen requirement and the gas mixture including nitric oxide from the receptacle to the patient.

The pulmonary artery pressure sensor can be configured to monitor the right heart.

The pulmonary artery pressure sensor can also be configured to monitor the left heart.

The pulmonary artery pressure sensor can be a wireless device.

Other features, objects, and advantages will be apparent from the description, drawings, and claims.

### BRIEF DESCRIPTION OF THE DRAWINGS

FIG. 1 is a schematic showing an embodiment of the claimed method.

FIG. 2 is an illustration of a receptacle.

FIGS. 3a) through c) are illustrations of a system including a receptacle.

FIG. 4 is a drawing depicting a system including a receptacle.

FIG. 5 is a graph showing nitric oxide and nitrogen dioxide concentrations as a function of time in comparison to a ventilator flow rate.

FIG. 6 is a graph showing nitric oxide and nitrogen dioxide concentrations as a function of time in comparison to a ventilator flow rate.

FIG. 7 is a graph showing nitric oxide concentration as a function of time in comparison to a ventilator flow rate.

FIG. 8 is a graph showing nitric oxide concentration as a function of time in comparison to a ventilator flow rate.

FIG. 9 is a graph showing nitric oxide concentration as a function of time in comparison to a ventilator flow rate.

FIG. 10 is a graph showing nitric oxide concentration as a function of time.

FIG. 11 is a graph showing a method of monitoring oxygen levels and monitoring pulmonary artery pressure.

### DETAILED DESCRIPTION

Some disorders or physiological conditions that require supplemental oxygen can be mediated by inhalation of nitric oxide. The use of low concentrations of inhaled nitric oxide can prevent, reverse, or limit the progression of disorders which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism and idiopathic or primary pulmonary hypertension or chronic hypoxia. Advantageously, nitric oxide can be generated and delivered in the absence of harmful side products, such as nitrogen dioxide. The nitric oxide can be generated at a concentration suitable for delivery to a mammal in need of treatment such that supplemental oxygen is administered to achieve a target effect while minimizing oxidative damage to a patient's tissues.

When delivering nitric oxide (NO) for therapeutic use to a mammal, it is also important to avoid delivery of nitrogen dioxide (NO₂) to the mammal. Nitrogen dioxide (NO₂) can be formed by the oxidation of nitric oxide (NO) with oxygen (O₂). The rate of formation of nitrogen dioxide (NO₂) can be proportional to the oxygen (O₂) concentration multiplied by the square of the nitric oxide (NO) concentration. A NO delivery system can convert nitrogen dioxide (NO₂) to nitric oxide (NO). Additionally, nitric oxide can form nitrogen dioxide at increased concentrations.

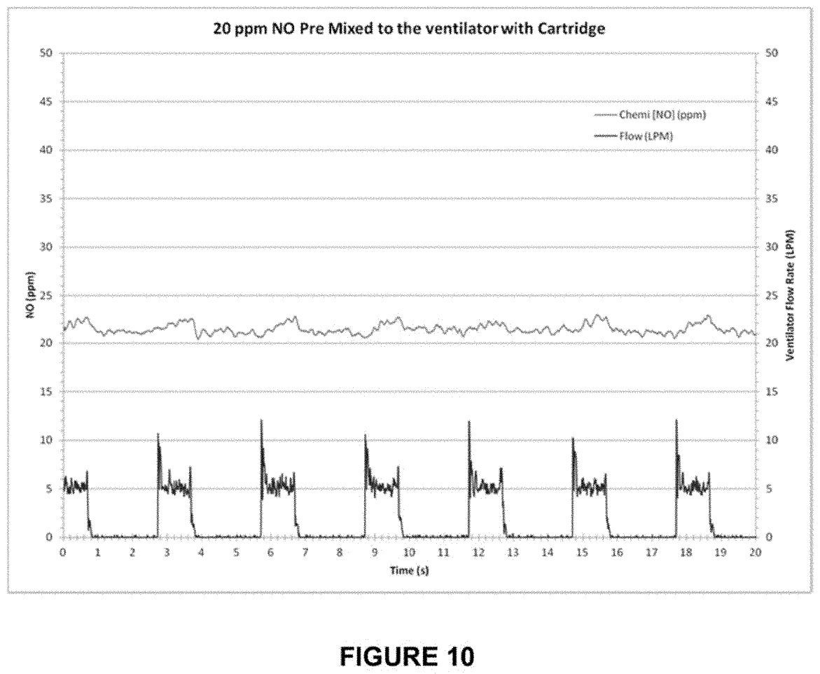

Referring to FIG. 10, a method of modulating oxygen saturation levels can include measuring oxygen saturation levels in a patient administering inhaled nitric oxide, adjusting the dose of oxygen in real time to a second dose based on the inhaled nitric oxide determining a first oxygen requirement to address an oxygen deficiency, determining a reduced oxygen requirement based on the generated nitric oxide, and delivering a dose of supplemental oxygen based on the reduced oxygen requirement and the gas mixture including nitric oxide from the receptacle to the patient. Adjusting the dose includes titrating the dose of oxygen in real time.

The method can also include mixing a first gas including oxygen and a second gas including a nitric oxide-releasing agent within a receptacle to form a gas mixture, wherein the receptacle includes an inlet, an outlet and a reducing agent and contacting the nitric oxide-releasing agent in the gas mixture with the reducing agent to generate nitric oxide.

The method of modulating oxygen saturation levels can also include measuring oxygen saturation levels in a patient, determining a first dose of oxygen to address an oxygen deficiency, mixing a first gas including oxygen and a second gas including a nitric oxide, determining a second dose of oxygen based on an amount of nitric oxide to be co-administered with the oxygen, wherein the second dose is lower than the first dose; and delivering the gas mixture including nitric oxide from the receptacle to the patient.

Situations Requiring Supplemental Oxygen

The administration of supplemental oxygen is an essential element of appropriate management for a wide range of clinical conditions, spanning different medical and surgical specialities. In general, the clinical goals of oxygen therapy are to treat hypoxemia, decrease the work of breathing and/or decrease myocardial work. The most common reasons for oxygen therapy to be initiated include acute hypoxemia such as that caused by shock, asthma, pneumonia or heart failure, ischemia such as cause by myocardial infarction, an abnormality in the quality or type of haemoglobin, acute blood loss in trauma or cyanide poisoning. A patient's need for oxygen therapy is based on a specific clinical condition. Oxygen therapy is prescribed for patients unable to get enough oxygen independently, often because of a lung condition that prevents the lings from absorbing oxygen, including COPD, pneumonia, asthma, dysplasia (or underdeveloped lungs in newborns), heart failures, cystic fibrosis, sleep apnea, lung disease, or trauma to the respiratory system.

Oxygen therapy is prescribed for both acute (short term) and chronic (long term) conditions and diseases. Short-term oxygen is usually prescribed for severe pneumonia, several asthma, respiratory distress syndrome (RDS) or bronchopulmonary dysplasia (BPD) in premature babies. Pneumonia involves an infection that causes a lung's air sacs to become inflamed. This prevents the air sacs from moving enough oxygen to the blood.

In a severe asthma attack, the airways become inflamed and narrowed. While most people with asthma can manage their symptoms, a severe asthma attack can require hospitalization and oxygen therapy. Finally, premature babies may receive extra oxygen through a nasal continuous positive airway pressure (NCPAP) machine or a ventilator, or through a nasal tube.

Long-term oxygen therapy can be used for certain conditions such as chronic obstructive pulmonary disease (COPD), pulmonary fibrosis, cystic fibrosis (CF), emphysema, chronic bronchitis, alpha 1 antitrypsin deficiency, and sleep-related breathing disorders. COPD is a progressive disease in which damage to the air sacs prevents them from moving enough oxygen into the bloodstream. “Progressive” means the disease gets worse over time.

CF is an inherited disease of the secretory glands, including the glands that make mucus and sweat. People who have CF have thick, sticky mucus that collects in their airways. The mucus makes it easy for bacteria to grow. This leads to repeated, serious lung infections. Over time, these infections can severely damage the lungs.

Emphysema is diagnosed when the small air sacs in the lungs gradually become compromised and the damage makes it harder to breathe normally. Those with emphysema often become short of breath on a regular basis. However, supplemental oxygen can help provide some relief by increasing blood oxygen levels and making oxygen distribution easier on the body.

Chronic bronchitis can also be caused by cigarette smoke and harmful toxins and pollutants breathed in over time. The disease, which will get worse over time, is characterized by a constant cough and large amount of mucus. When caught early, the disease can then be managed.

Alpha 1 antitrypsin deficiency is a genetic disorder that can lead to breathing problems at a young age and eventually develop into emphysema or Chronic Obstructive Pulmonary Disease (COPD). The Alpha 1 Antitrypsin enzyme is found in the lungs and bloodstream and is meant to prevent inflammation and its effects in the lungs. When a patient's body lacks enough of this enzyme, it can lead to emphysema and make it difficult to breathe. Supplemental oxygen, along with bronchodilators and pulmonary rehabilitation, are common treatments.

Sleep-related breathing disorders that lead to low levels of oxygen in the blood during sleep, such as sleep apnea and late stage heart failure can also require oxygen therapy. This is a condition in which the heart is unable to pump enough oxygen-rich blood to meet the body's needs.

Measuring Oxygen Saturation Levels

In patients in need of oxygen therapy, the first step is to measure the patient's oxygen saturation levels. This measurement is typically conducted using pulse oximetry. A pulse oximeter is a medical device that indirectly monitors the oxygen saturation of a patient's blood (as opposed to measuring oxygen saturation directly through a blood sample) and changes in blood volume in the skin. The pulse oximeter may be incorporated into a multi-parameter patient monitor. Most monitors also display the pulse rate. Portable, battery-operated pulse oximeters are also available for transport or home blood-oxygen monitoring.

In pulse oximetry, a transdermal sensor is placed on a thin part of the patient's body such as a fingertip or earlobe, or in the case of an infant, across a foot. The device passes two wavelengths of light through the body part to a photodetector. The photodetector measures the changing absorbance at each of the wavelengths, allowing it to determine the absorbances due to the pulsing arterial blood. Pulse oximetry is available for certain smartphones.

Alternatively, reflectance pulse oximetry may be used, which does not require selecting a thin section of the person's body and is therefore well suited to more universal applications, such as the feet, forehead and chest. However, this method also has so limitations. Vasodilation and pooling of venous blood in the head due to compromised venous return to the heart, as occurs with congenital cyanotic heart disease patients, or in patients in the Trendelenburg position, can cause a combination of arterial and venous pulsations in the forehead region and lead to spurious SpO2 (Saturation of peripheral oxygen) results.

Real Time Monitoring

Oxygen levels can be monitored in a variety of ways. For example, oxygen levels can be monitored by a wireless monitoring system. The wireless monitoring system is typically composed of three components: a telemetric implant (including an implantable pulmonary artery sensor), a monitoring unit, and the database management system (e.g. a Patient Electronics System) for internet-based worldwide access. The wireless monitoring system can be used to monitor the left heart (left atrium or left ventricle), right heart (right atrium or right ventricle), or both.

There are generally two categories of implants: implantable hemodynamic monitors implanted adjunct to a planned thoracic surgery and implants that are delivered percutaneously via catheter-based techniques in either the pulmonary artery (PA) or left atrium during a stand-alone procedure. The PA sensor is about the size of small paper clip and has a thin, curved wire at each end. This sensor does not require any batteries or wires.

The delivery system is a long, thin, flexible tube (catheter) that moves through the blood vessels and is designed to release the implantable sensor in the far end of the pulmonary artery.

The Patient Electronics System includes the electronics unit, antenna and pillow. Together, the components of the Patient Electronics System read the PA pressure measurements from the sensor wirelessly and then transmit the information to the doctor. The antenna is for example, paddle-shaped and is pre-assembled inside a pillow to make it easier and more comfortable for the patient to take readings.

The sensor monitors the pressure in the pulmonary artery. Patients take a daily reading from home or other non-clinical locations using the Patient Electronics System which sends the information to the doctor. After analyzing the information, the doctor may make medication changes to help treat the patient's heart failure.

One example of a system used to monitor pulmonary artery pressure is the CardioMEMS™ system. The CardioMEMS HF System can be used to wirelessly measure and monitor PA pressure and heart rate in New York Heart Association (NYHA) Class III heart failure patients who have been hospitalized for heart failure in the previous year. The PA pressure and heart rate are used by doctors for heart failure management and with the goal of reducing heart failure hospitalizations.

The CardioMEMS HF System is used by the doctor in the hospital or medical office setting to obtain and review PA pressure measurements. The patient uses the CardioMEMS HF System at home or other non-clinical locations to wirelessly obtain and send PA pressure and heart rate measurements to a secure database for review and evaluation by the patient's doctor.

Access to PA pressure data provides doctors with another way to better manage a patient's heart failure and potentially reduce heart failure-related hospitalizations. Reducing heart failure hospitalizations has a direct impact on a patient's well-being. In a clinical study in which 550 participants had the device implanted, there was a clinically and statistically significant reduction in heart failure-related hospitalizations for the participants whose doctors had access to PA pressure data. Additionally, there were no device or system-related complications or pressure sensor failures through six months.

The system can measure pulmonary artery (PA) pressure. A pulmonary artery pressure sensor can be implanted in a pulmonary artery, and the sensor can transmit data through an electronic system. As a result, right ventricular pressure or left ventricular pressure, or both, can be evaluated.

The implanted device can collect data for pulmonary artery pressure (mPAP), systolic pulmonary artery pressure (sPAP), diastolic pulmonary artery pressure (dPAP), heart rate (HR), and/or cardia output (CO) through a sensor pressure based algorithm. The data can be collected in real time.

Use of the CardioMEMS™ in the MRI environment has been shown to be feasible and produce valuable adjunctive information. The ability to simultaneously assess volumetric and pressure responses to hemodynamic challenges has been demonstrated. Of interest is the response of the ventricular vascular coupling ratio to iNO and dobutamine.

In iNO non responders, there was minimal change to ventricular vascular coupling (VVC), but patients are more responsive to changes in dobutamine.

An example of wireless monitoring is described in “A Study to Explore the Feasibility and Safety of Using Cardiomems HF System in PAH Patients,”Am. J. Respir. Crit. Care. Med. 191; 2015-A5529.

A similar wireless monitoring system can be used to monitor the right heart (right atrium or right ventricle). It is crucial to note that the two sides of the heart (left and right side) can fail independently of each other, and each event has its own causes and effects

The heart has two jobs: to collect returning, “used” blood and pump it into the lungs to be enriched with oxygen, and to take oxygen-rich blood from the lungs and pump it out to the rest of the body. The left ventricle is by far the larger of the two halves of the heart, because it does the difficult job of pumping blood out to the entire body. It draws the blood from the left lung where it has been filled with fresh oxygen. The pumping of this side of the heart sends the blood out to all the body's organs and extremities, which need the oxygen to live and work. As oxygen is depleted from the blood, it returns to the heart on the right side. The right ventricle pumps the blood back to the lungs to start the process over. Both the left and right ventricles' jobs are necessary for people to live—and either or both can be interrupted by heart failure.

Heart failure occurs when one or both sides of the heart have difficult pumping (or difficulty relaxing between pumps). This can be caused by many things, from a blood clot or heart attack to congenital factors. However, heart failure has different effects, depending on which side it strikes.

In left-sided heart failure, the heart can no longer adequately bring in fresh blood from the lung and pump it out to the body. This causes blood to back up and pool in the left lung. Shortness of breath, heaviness in the chest and difficulty breathing are common signs of left-sided heart failure.

Right-sided heart failure often occurs in response to left-sided failure. The right ventricle becomes overworked and fails in turn. If right-sided heart failure occurs on its own, blood returning from the body becomes backed up.

A PA sensor for the right heart can similarly be designed for implantation. The PA sensor for the right heart can also be configured to be about the size of small paper clip and have a thin, curved wire at each end. This sensor does not require any batteries or wires. The delivery system for the right heart can also have a long, thin, flexible tube (catheter) that moves through the blood vessels and is designed to release the implantable sensor in the far end of the pulmonary artery.

The Patient Electronics System for a right heart can also include the electronics unit, antenna and pillow. Together, the components of the Patient Electronics System read the PA pressure measurements from the sensor wirelessly and then transmit the information to the doctor. The antenna is for example, paddle-shaped and is pre-assembled inside a pillow to make it easier and more comfortable for the patient to take readings.

The sensor monitors for the right heart can also monitor the pressure in the pulmonary artery. Patients take a daily reading from home or other non-clinical locations using the Patient Electronics System which sends the information to the doctor. After analyzing the information, the doctor may make medication changes to help treat the patient's heart failure.

Determining Supplemental Oxygen Requirement

Based on the measured oxygen saturation levels and the diagnosis of the patient's condition, a medical provider such as a physician then determines and selects an effective dose of supplemental oxygen to administer to a patient. A healthy patient's baseline oxygen saturation levels are typically 95-100 percent. If a patient's oxygen saturation levels are below 90 percent, supplemental oxygen therapy is usually required, and the appropriate dose of supplemental oxygen is determined based on the deficiency.

In a patient with acute respiratory illness (e.g., influenza) or breathing difficulty (e.g, an asthma attack), an SpO2 of 92% or less may indicate a need for oxygen supplementation. In a patient with stable chronic disease (e.g., COPD), an SpO2 of 92% or less should prompt referral for further investigation of the need for long-term oxygen therapy.

For example, if the measure oxygen saturation level is 80 percent, a typical dose of supplemental oxygen for low flow delivery devices is 1-6 L/min via nasal cannula and 5-6 L/min via oxygen mask. High flow delivery devices can offer a typical dose of about 30 L/min, or higher.

Depending on the diagnosed condition, the goal of supplemental oxygen is generally to maintain a PaO₂ of 55-60 mmHg, which corresponds to SpO₂ of about 90%. Higher concentrations of oxygen can blunt the hypoxic ventilatory drive, which may precipitate hypoventilation and CO₂ retention.

The fraction of inspired oxygen (FiO₂) is the fraction or percentage of oxygen in the space being measured. Medical patients experiencing difficulty breathing are provided with oxygen-enriched air, which means a higher-than-atmospheric FiO₂. Natural air includes 20.9% oxygen, which is equivalent to FiO₂ of 0.209. Oxygen-enriched air has a higher FiO₂ than 0.21, up to 1.00, which means 100% oxygen. FiO₂ is typically maintained below 0.5 even with mechanical ventilation, to avoid oxygen toxicity. If a patient is wearing a nasal cannula or a simple face mask, each additional liter of oxygen adds about 4% to their FiO₂ (for example, a patient with a nasal cannula with 2 L of oxygen attached would have an FiO₂ of 21%+8%=29%). The ratio of partial pressure arterial oxygen and fraction of inspired oxygen, sometimes called the Carrico index, is a comparison between the oxygen level in the blood and the oxygen concentration that is breathed.

Potential Adverse Effects of Oxygen

In general, oxygen therapy is safe and effective. The net effect of oxygen therapy is to reverse hypoxaemia and the benefits generally outweigh the risks. However, hazards of oxygen therapy that a clinician must recognize include oxygen toxicity and CO₂ retention. While there is a growing acknowledgment of oxygen as a drug with specific biochemical and physiologic actions in a distinct range of effective doses, there are also well-defined adverse effects at high doses.

Patients exposed to inspiratory oxygen fraction (FiO₂)>28% may experience oxygen toxicity, particularly if the exposure is prolonged. Oxygen toxicity is related to free radicals. The major end product of normal oxygen metabolism is water. Some oxygen molecules, however, are converted into highly reactive radicals, which include superoxide anions, perhydroxy radicals and hydroxyl radicals, and are toxic to alveolar and tracheobronchial cells.

Pathophysiological changes include decreased lung compliance, reduced inspiratory airflow, decreased diffusing capacity and small airway dysfunction. While these changes are well recognised in the acute care setting of mechanically ventilated patients receiving FiO₂>50%, little is known about the long-term effect of low flow (24-28%) oxygen. It is widely accepted that the increased survival and quality-of-life benefits of long-term oxygen therapy outweigh the possible risks.

Indeed, there are certain situations in which oxygen therapy is known to have a negative impact on a patient's condition. For example, in a patient who is suffering from paraquat poisoning, oxygen can increase the toxicity. Moreover, oxygen therapy is typically not recommended for patients who have suffered pulmonary fibrosis or other lung damage resulting from bleomycin treatment.

In addition, high levels of oxygen given to infants typically causes blindness by promoting overgrowth of new blood vessels in the eye obstructing sight. This is termed retinopathy of prematurity (ROP). See, e.g., O. D. Saugstaad, Journal of Perinatology (2006) 26, S46-S50.

Exacerbations of Chronic Obstructive Pulmonary Disease COPD

Patients of chronic obstructive pulmonary disease (COPD) often have chronic hypoxaemia with or without CO₂ retention. Oxygen in this situation is required until the exacerbation is settled. While a high FiO₂ of up to 100% can be initially administered in case hypoxemia is severe, it is soon tapered to around 50-60% FiO₂.

As previously discussed, the goal of supplemental oxygen is to maintain a PaO₂ of 55-60 mmHg, which corresponds to SpO2 of about 90%, since higher concentrations of oxygen can blunt the hypoxic ventilatory drive, which may precipitate hypoventilation and CO₂ retention. Thus, it is advisable to use a regulated flow device such as a venti mask, which guarantees oxygen delivery to a reasonable extent. Once the patient is stabilized, one can shift to nasal prongs—a device that is more comfortable and acceptable to the patient.

Acute Severe Bronchial Asthma

Patients with acute severe asthma or status asthamticus have severe airway obstruction and inflammation. They are generally hypoxemic. Arterial blood sample is immediately obtained and oxygen is started via nasal cannula or preferably via a face mask at flow rate of 4-6 L/min to achieve FiO₂ of 35 to 40%. Higher flow is unlikely to improve oxygenation. Flow rate is adjusted to maintain a PaO₂ of about 80 mmHg or near normal value. Concurrent bronchial hygiene and administration of intravenous fluids, bronchodilators and corticosteroids should alleviate the problems in most of the situations. Administration of sedatives and tranquilizers must be avoided. Sedatives may precipitate CO₂ retention not only in patients with COPD, but also asthma. Assisted ventilation is required in case there is persistence of hypoxemia and/or precipitation of hypercapnia.

Hyperoxia

Oxidative cell injury involves the modification of cellular macromolecules by reactive oxygen intermediates (ROI), often leading to cell death.

Hyperoxia injures cells by virtue of the accumulation of toxic levels of ROI, including H₂O₂ and the superoxide anion (O₂—), which are not adequately scavenged by endogenous antioxidant defenses. These oxidants are cytotoxic and have been shown to kill cells via apoptosis, or programmed cell death. If hyperoxia-induced cell death is a result of increased ROI, then O₂ toxicity should kill cells via apoptosis. It has been discovered that hyperoxia kills cells via necrosis, not apoptosis. In contrast, lethal concentrations of either H₂O₂ or O₂— cause apoptosis. Paradoxically, apoptosis is a prominent event in the lungs of animals injured by breathing 100% O₂. These data indicate that O₂ toxicity is somewhat distinct from other forms of oxidative injury and suggest that apoptosis in vivo is not a direct effect of O₂.

Exposure to high oxygen concentration causes direct oxidative cell damage through increased production of reactive oxygen species. In vivo oxygen-induced lung injury is well characterized in rodents and has been used as a valuable model of human respiratory distress syndrome. Hyperoxia-induced lung injury can be considered as a bimodal process resulting (1) from direct oxygen toxicity and (2) from the accumulation of inflammatory mediators within the lungs. Both apoptosis and necrosis have been described in alveolar cells (mainly epithelial and endothelial) during hyperoxia. While the in vitro response to oxygen seems to be cell type-dependent in tissue cultures, it is still unclear which are the death mechanisms and pathways implicated in vivo. Even though it is not yet possible to distinguish unequivocally between apoptosis, necrosis, or other intermediate form(s) of cell death, a great variety of strategies has been shown to prevent alveolar damage and to increase animal survival during hyperoxia.

Oxygen administration can cause structural damage to the lungs. Both proliferative and fibrotic changes of oxygen toxicity have been shown at autopsy on COPD patients treated with long term oxygen. But there is no significant effect of these changes on clinical course or survival of these patients. Most of the structural damage attributable to hyperoxia results from high FiO₂ administration in acute conditions.

With prolonged oxygen therapy there is increase in blood oxygen level, which suppresses peripheral chemoreceptors; depresses ventilator drive and increase in PCO₂. high blood oxygen level may also disrupt the ventilation: perfusion balance (V/Q) and cause an increase in dead space to tidal volume ratio and increase in PCO₂. Therefore, oxygen therapy may accentuate hypoventilation in patients with COPD. This may include hypercapnia and carbon dioxide narcosis. Prehospital hyperoxia from excessive oxygen administration in COPD patients is shown to be dangerous.

An FiO2>0.50 presents a significant risk of absorption atelactasis. N₂ is most plentiful gas in both the alveoli and blood. Breathing high level of O₂ depletes body N₂ levels. As blood N₂ level decreases, total pressure of venous gases rapidly decreases. Under these conditions, gases within any body cavity rapidly diffuse into venous blood leading to absorption atelactasis. Risk of absorption atelactasis is greatest in patients breathing at low tidal volumes as a result of sedation, surgical pain or central nervous system (CNS) dysfunction. See, e.g., Singh, et al., Supplemental oxygen therapy: Important considerations in oral and maxillofacial surgery, Natl. J. Maxillofac. Surg., 2(1):10-14, January-June 2011.

Role of NO

Nitric oxide is an important signaling molecule in pulmonary vessels. Nitric oxide can moderate pulmonary hypertension caused by elevation of the pulmonary arterial pressure. Inhaling low concentrations of nitric oxide, for example, in the range of 0.01-100 ppm can rapidly and safely decrease pulmonary hypertension in a mammal by vasodilation of pulmonary vessels.

NO has been implicated as both a prooxidant and an antioxidant. One might anticipate, therefore, that the addition of NO in the presence of high inspired O2 might modify the overall response to the high O2 exposure. For example, high O2 increases superoxide production, and superoxide and NO react spontaneously to form peroxynitrite, which can be toxic. Furthermore, oxygen and NO readily combine to form NO₂, which can also be toxic. On the other hand, NO can react with lipid peroxyl radicals to prevent lipid peroxidation, and this might help thwart the increase in lipid peroxidation associated with oxygen toxicity. Furthermore, NO can inhibit neutrophil accumulation and activation. It has been shown that, when endogenous NO production was blocked in neonatal rats, which are relatively O₂-tolerant with Nω-nitro-1-arginine methyl ester, significantly fewer survived exposure to >95% O₂ compared with control rats, suggesting that endogenous NO has some protective effect.

Inhaled NO was shown to increase survival in high O₂ exposure in rats. The impact of adding NO to high inspired O₂ is clinically relevant because many patients with various forms of acute lung injury, such as adult respiratory distress syndrome, persistent pulmonary hypertension of the newborn caused by meconium aspiration, and so forth, are being treated with inhaled NO while receiving very high fractions of inspired O₂.

In short, using NO allows one to use a reduced amount of supplemental oxygen, thereby reducing oxidative stress, while providing the necessary oxygen enhancement.

Potential Toxicity of NO

Studies have shown that short-term exposure to inhaled NO, O2 or O2+NO increases lung collagen accumulation in neonatal piglets. This may be because NO, unlike O2 or O2+NO, does not induce a concurrent increase in pulmonary matrix degradation. Indeed the increase in lung collagen content found with NO exposure appeared potentially reversible as demonstrated by a significant decline after a 3-day recovery period in RA. The increase in lung collagen accumulation observed with NO represents a finding that NO may have the potential to induce pulmonary fibrosis. Ekekezie, High-dose Inhaled Nitric Oxide and Hyperoxia Increases Lung Collagen Accumulation in Piglets, Biology of the Neonate, 78(3) (2000).

Hydrogen Supplement

Hydrogen gas can act as an antioxidant and is a free radical scavenger. Hydrogen is the most abundant chemical element in the universe, but is seldom regarded as a therapeutic agent. Recent evidence has shown that hydrogen is a potent antioxidative, antiapoptotic and anti-inflammatory agent and so may have potential medical applications in cells, tissues and organs.

Using a mixture of NO and hydrogen gases for inhalation can be useful, for example, during planned coronary interventions or for the treatment of ischemia-reperfusion (I/R) injury. In short, inhaled NO suppresses the inflammation in I/R tissues and hydrogen gas eliminates the adverse by-products of NO exposure, peroxynitrite.

However until applicants' discovery, there has not been a successful combination of hydrogen gas with breathing gas using the claimed apparatus and methods.

Applicants have discovered that NO's effect as an antioxidant may be enhanced by eliminating highly reactive by-products of NO inhalation such as peroxynitrite, by adding H2 to inhaled NO gas. Specifically, Applicants found that 1) mice with intratracheal administration of LPS exhibited significant lung injury, which was significantly improved by 2% H₂ and/or 20 ppm NO treatment for 3 hours starting at 5 minutes or 3 hours after LPS administration; 2) H₂ and/or NO treatment inhibited LPS-induced pulmonary early and late NF-κB activation; 3) H₂ and/or NO treatment down-regulated the pulmonary inflammation and cell apoptosis; 4) H₂ and/or NO treatment also significantly attenuated the lung injury in polymicrobial sepsis; and 5) Combination therapy with subthreshold concentrations of H₂ and NO could synergistically attenuate LPS- and polymicrobial sepsis-induced lung injury. In conclusion, these results demonstrate that combination therapy with H₂ and NO could more significantly ameliorate LPS- and polymicrobial sepsis-induced ALI, perhaps by reducing lung inflammation and apoptosis, which may be associated with the decreased NF-κB activity.

Studies have shown that hydrogen gas exhibits cytoprotective effects and transcriptional alterations, and can selectively reduce the generation of hydroxyl radicals and peroxynitrite, thereby protecting the cells against oxidant injury. Yokota, Molecular hydrogen protects chrondrocytes from oxidative stress and indirectly alters gene expressions through reducing peroxynitrite derived from nitric oxide, Medical Gas Research 2015.

In an acute rat model in which oxidative stress was induced in the brain by focal FiOischemia-reperfusion (I/R), inhaled hydrogen gas markedly suppressed the associated brain injury. Thus it was suggested that administration of hydrogen gas by inhalation may serve as an effective therapy for ischemia-reperfusion, and based on the ability of hydrogen gas to rapidly diffuse across membranes, it can even protect ischemic tissues against oxidative damage. Ohsawa I, et al., Hydrogen acts as a therapeutic antioxidant by selectively reducing cytotoxic oxygen radicals. Nat Med 13: 688-694, 2007.

Breathing NO plus hydrogen gas was also found to reduce cardiac injury and augment recovery of the left ventricular function, by elimination of the nitrotyrosine produced by NO inhalation alone. See, e.g., Shinbo, et al., “Breathing nitric oxide plus hydrogen has reduced ischemia-reperfusion injury and nitrotyrosine production in murine heart,” Am J. Physiol Heart Circ Physiol., 305: H542-H550, 2013. In addition, data has indicated that combination therapy with hydrogen gas and NO can effectively attenuate LPS-induced lung inflammation and injury in mice. Liu, et al, “Combination therapy with NO and H₂ in ALI.”

There are several methods to administer hydrogen, such as inhalation of hydrogen gas, aerosol inhalation of a hydrogen-rich solution, drinking hydrogen dissolved in water, injecting hydrogen-rich saline (HRS) and taking a hydrogen bath. Drinking hydrogen solution (saline/pure water/other solutions saturated with hydrogen) may be more practical in daily life and more suitable for daily consumption. Shen, et al., “A review of experimental studies of hydrogen as a new therapeutic agent in emergency and critical care medicine.” Medical Gas Research, 2014. Molecular hydrogen diffuses rapidly across cell membranes, reduces reactive oxygen species, including hydroxyl radicals and peroxynitrite, and suppresses oxidative stress-induced injury in several organs with no known toxicity. Fu, et al., Molecular hydrogen is protective against 6-hydroxydopamine-induced nigrostriatal degeneration in a rat model of Parkinson's disease. Neurosci. Lett. 2009.

Supplemental hydrogen may also prove effective in reducing oxidative stress when combined with NO+O2(H+NO+O2), or with O2(H+O2).

Administering Supplemental Oxygen

Supplemental oxygen and NO can be administered by titration. NO can be administered by titration. Titration is a method or process of administering a dose of compound such as NO until a visible or detectable change is achieved.

Any suitable system can be used to deliver NO. NO can be administered by titration. As previously discussed, titration is a method or process of determining the concentration of a dissolved substance in terms of the smallest amount of reagent of known concentration required to bring about a given effect in reaction with a known volume of the test solution.

In one embodiment, a nitric oxide delivery system can include a cartridge. A cartridge can include an inlet and an outlet. A cartridge can convert a nitric oxide-releasing agent to nitric oxide (NO). A nitric oxide-releasing agent can include one or more of nitrogen dioxide (NO₂), dinitrogen tetroxide (N₂O₄) or nitrite ions (NO₂—). Nitrite ions can be introduced in the form of a nitrite salt, such as sodium nitrite.

A cartridge can include a reducing agent or a combination of reducing agents. A number of reducing agents can be used depending on the activities and properties as determined by a person of skill in the art. In some embodiments, a reducing agent can include a hydroquinone, glutathione, and/or one or more reduced metal salts such as Fe(II), Mo(VI), NaI, Ti(III) or Cr(III), thiols, or NO₂—. A reducing agent can include 3,4 dihydroxy-cyclobutene-dione, maleic acid, croconic acid, dihydroxy-fumaric acid, tetra-hydroxy-quinone, p-toluene-sulfonic acid, tricholor-acetic acid, mandelic acid, 2-fluoro-mandelic acid, or 2,3,5,6-tetrafluoro-mandelic acid. A reducing agent can be safe (i.e., non-toxic and/or non-caustic) for inhalation by a mammal, for example, a human. A reducing agent can be an antioxidant. An antioxidant can include any number of common antioxidants, including ascorbic acid, alpha tocopherol, and/or gamma tocopherol. A reducing agent can include a salt, ester, anhydride, crystalline form, or amorphous form of any of the reducing agents listed above. A reducing agent can be used dry or wet. For example, a reducing agent can be in solution. A reducing agent can be at different concentrations in a solution. Solutions of the reducing agent can be saturated or unsaturated. While a reducing agent in organic solutions can be used, a reducing agent in an aqueous solution is preferred. A solution including a reducing agent and an alcohol (e.g. methanol, ethanol, propanol, isopropanol, etc.) can also be used.

A cartridge can include a support. A support can be any material that has at least one solid or non-fluid surface (e.g. a gel). It can be advantageous to have a support that has at least one surface with a large surface area. In preferred embodiments, the support can be porous or permeable. One example of a support can be surface-active material, for example, a material with a large surface area that is capable of retaining water or absorbing moisture. Specific examples of surface active materials can include silica gel or cotton. The term “surface-active material” denotes that the material supports an active agent on its surface.

A support can include a reducing agent. Said another way, a reducing agent can be part of a support. For example, a reducing agent can be present on a surface of a support. One way this can be achieved can be to coat a support, at least in part, with a reducing agent. In some cases, a system can be coated with a solution including a reducing agent. Preferably, a system can employ a surface-active material coated with an aqueous solution of antioxidant as a simple and effective mechanism for making the conversion. Generation of NO from a nitric oxide-releasing agent performed using a support with a reducing agent can be the most effective method, but a reducing agent alone can also be used to convert nitric oxide-releasing agent to NO.

In some circumstances, a support can be a matrix or a polymer, more specifically, a hydrophilic polymer. A support can be mixed with a solution of the reducing agent. The solution of reducing agent can be stirred and strained with the support and then drained. The moist support-reducing agent mixture can be dried to obtain the proper level of moisture. Following drying, the support-reducing agent mixture may still be moist or may be dried completely. Drying can occur using a heating device, for example, an oven or autoclave, or can occur by air drying.

In general, a nitric oxide-releasing agent can be converted to NO by bringing a gas including the nitric oxide-releasing agent in contact with a reducing agent. In one example, a gas including a nitric oxide-releasing agent can be passed over or through a support including a reducing agent. When the reducing agent is ascorbic acid (i.e. vitamin C), the conversion of nitrogen dioxide to nitric oxide can be quantitative at ambient temperatures.

The generated nitric oxide can be delivered to a mammal, which can be a human. To facilitate delivery of the nitric oxide, a system can include a patient interface. Examples of a patient interface can include a mouth piece, nasal cannula, face mask, fully-sealed face mask or an endotracheal tube. A patient interface can be coupled to a delivery conduit. A delivery conduit can include a ventilator or an anesthesia machine.

### Modulating Hormesis

A method of providing NO can include administering exogenous NO to modulate the hormesis characteristics of NO. Hormesis in this instance refers to the temporal and dose dependency related to the stimulatory versus inhibitory response to NO. For example, NO stimulates HIF for 30 minutes at low dose during hypoxia. It becomes inhibitory at high doses and after 30 minutes. This suggests that it would be effective to lower doses 0.1 to 5 ppm for up to 30 minutes repeated at intervals rather than high dose continuous delivery, for example.

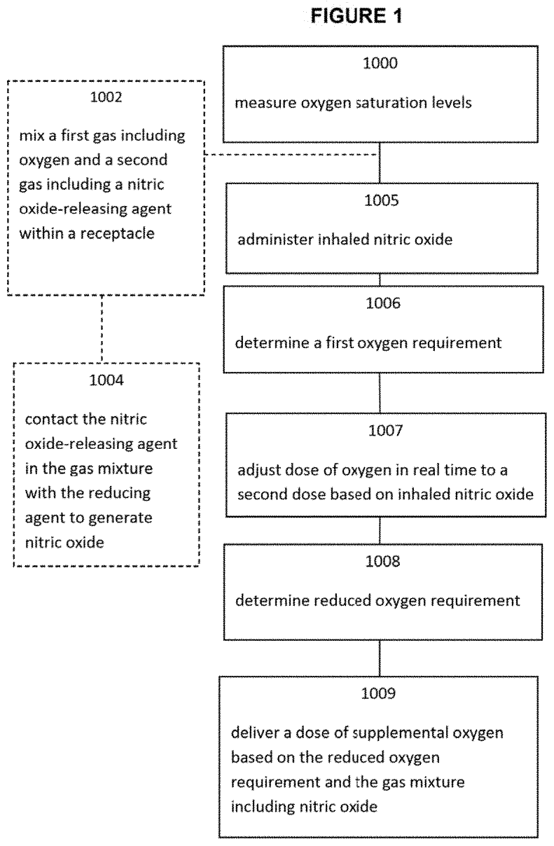

FIG. 1 shows an embodiment of the invention. The method includes measuring oxygen levels in a patient (1000) and administering inhaled nitric oxide (1005). In certain embodiments, the method can optionally include mixing a first gas including oxygen gas and a second gas including a nitric-oxide releasing agent within a cartridge (1003) and then contacting the nitric oxide-releasing agent with the reducing agent to generate nitric oxide (1004). The method can further include determining a first oxygen requirement (1006) based on a patient's condition or disease state, for example. Upon determining an oxygen requirement, a clinician such as a physician or other professional or person operating in a health care capacity, can then adjust the dose of oxygen in real time to a second dose based on the inhaled nitric oxide (1007). The clinician can determine a reduced oxygen requirement (1008) in view of the inhaled nitric oxide, either before or after the dose of oxygen is adjusted to a second dose or titrated until a target level of oxygen is reached. After a reduced oxygen requirement is determined or adjusted, a clinician can deliver a dose of supplemental oxygen based on the reduced oxygen requirement and the gas mixture including nitric oxide (1009).

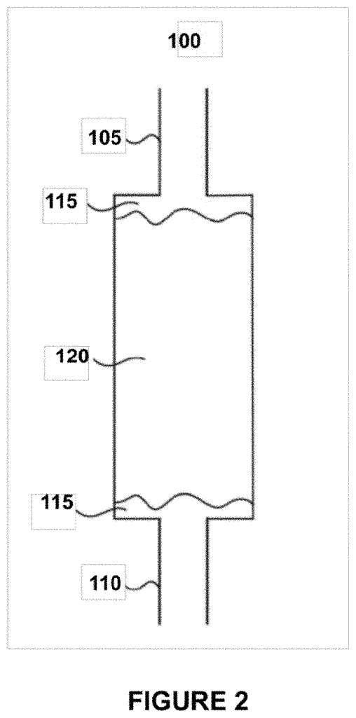

FIG. 2 illustrates one embodiment of a cartridge for generating NO by converting a nitric oxide-releasing agent to NO. The cartridge 100 can include an inlet 105 and an outlet 110. A cartridge can be inserted into and removed from an apparatus, platform or system. Preferably, a cartridge is replaceable in the apparatus, platform or system, and more preferably, a cartridge can be disposable. Screen and glass wool 115 can be located at either or both of the inlet 105 and the outlet 110. The remainder of the cartridge 100 can include a support. In a preferred embodiment, a receptacle 100 can be filled with a surface-active material 120. The surface-active material 120 can be soaked with a saturated solution of antioxidant in water to coat the surface-active material. The screen and glass wool 115 can also be soaked with the saturated solution of antioxidant in water before being inserted into the cartridge 100.

In general, a process for converting a nitric oxide-releasing agent to NO can include passing a gas including a nitric oxide-releasing agent into the inlet 105. The gas can be communicated to the outlet 110 and into contact with a reducing agent. In a preferred embodiment, the gas can be fluidly communicated to the outlet 110 through the surface-active material 120 coated with a reducing agent. As long as the surface-active material remains moist and the reducing agent has not been used up in the conversion, the general process can be effective at converting a nitric oxide-releasing agent to NO at ambient temperature.

The inlet 105 may receive the gas including a nitric oxide-releasing agent from a gas pump that fluidly communicates the gas over a diffusion tube or a permeation cell. The inlet 105 also may receive the gas including a nitric oxide-releasing agent, for example, from a pressurized bottle of a nitric oxide-releasing agent. A pressurized bottle may also be referred to as a tank. The inlet 105 also may receive a gas including a nitric oxide-releasing agent can be NO₂ gas in nitrogen (N₂), air, or oxygen (O₂). A wide variety of flow rates and NO₂ concentrations have been successfully tested, ranging from only a few ml per minute to flow rates of up to 5,000 ml per minute.

The conversion of a nitric oxide-releasing agent to NO can occur over a wide range of concentrations of a nitric oxide-releasing agent. For example, experiments have been carried out at concentrations in air of from about 2 ppm NO₂ to 100 ppm NO₂, and even to over 1000 ppm NO₂. In one example, a cartridge that was approximately 6 inches long and had a diameter of 1.5-inches was packed with silica gel that had first been soaked in a saturated aqueous solution of ascorbic acid. The moist silica gel was prepared using ascorbic acid designated as A.C.S reagent grade 99.1% pure from Aldrich Chemical Company and silica gel from Fischer Scientific International, Inc., designated as S8 32-1, 40 of Grade of 35 to 70 sized mesh. Other sizes of silica gel can also be effective. For example, silica gel having an eighth-inch diameter can also work.

In another example, silica gel was moistened with a saturated solution of ascorbic acid that had been prepared by mixing 35% by weight ascorbic acid in water, stirring, and straining the water/ascorbic acid mixture through the silica gel, followed by draining. The conversion of NO₂ to NO can proceed well when the support including the reducing agent, for example, silica gel coated with ascorbic acid, is moist. In a specific example, a cartridge filled with the wet silica gel/ascorbic acid was able to convert 1000 ppm of NO₂ in air to NO at a flow rate of 150 ml per minute, quantitatively, non-stop for over 12 days.

A cartridge can be used for inhalation therapy. In addition to converting a nitric oxide-releasing agent to nitric oxide to be delivered during inhalation therapy, a cartridge can remove any NO₂ that chemically forms during inhalation therapy (e.g., nitric oxide that is oxidized to form nitrogen dioxide). In one such example, a cartridge can be used as a NO₂ scrubber for NO inhalation therapy that delivers NO from a pressurized bottle source. A cartridge may be used to help ensure that no harmful levels of NO₂ are inadvertently inhaled by the patient.

In addition, a cartridge may be used to supplement or replace some or all of the safety devices used during inhalation therapy in conventional NO inhalation therapy. For example, one type of safety device can warn of the presence of NO₂ in a gas when the concentration of NO₂ exceeds a preset or predetermined limit, usually 1 part per million or greater of NO₂. Such a safety device may be unnecessary when a cartridge is positioned in a NO delivery system just prior to the patient breathing the NO laden gas. A cartridge can convert any NO₂ to NO just prior to the patient breathing the NO laden gas, making a device to warn of the presence of NO₂ in gas unnecessary.

Furthermore, a cartridge placed near the exit of inhalation equipment, gas lines or gas tubing can also reduce or eliminate problems associated with formation of NO₂ that occur due to transit times in the equipment, lines or tubing. As such, use of a cartridge can reduce or eliminate the need to ensure the rapid transit of the gas through the gas plumbing lines that is needed in conventional applications. Also, a cartridge can allow the NO gas to be used with gas balloons to control the total gas flow to the patient.

Alternatively or additionally, a NO₂ removal cartridge can be inserted just before the attachment of the delivery system to the patient to further enhance safety and help ensure that all traces of the toxic NO₂ have been removed. The NO₂ removal cartridge may be a cartridge used to remove any trace amounts of NO₂. Alternatively, the NO₂ removal cartridge can include heat-activated alumina. A cartridge with heat-activated alumina, such as supplied by Fisher Scientific International, Inc., designated as ASOS-212, of 8-14 sized mesh can be effective at removing low levels of NO₂ from an air or oxygen stream, and yet, can allow NO gas to pass through without loss. Activated alumina, and other high surface area materials like it, can be used to scrub NO₂ from a NO inhalation line.

In another example, a cartridge can be used to generate NO for therapeutic gas delivery. Because of the effectiveness of a cartridge in converting nitric oxide-releasing agents to NO, nitrogen dioxide (gaseous or liquid) or dinitrogen tetroxide can be used as the source of the NO. When nitrogen dioxide or dinitrogen tetroxide is used as a source for generation of NO, there may be no need for a pressurized gas bottle to provide NO gas to the delivery system. By eliminating the need for a pressurized gas bottle to provide NO, the delivery system may be simplified as compared with a conventional apparatus that is used to deliver NO gas to a patient from a pressurized gas bottle of NO gas. A NO delivery system that does not use pressurized gas bottles may be more portable than conventional systems that rely on pressurized gas bottles.

In some delivery systems, the amount of nitric oxide-releasing agent in a gas can be approximately equivalent to the amount of nitric oxide to be delivered to a patient. For example, if a therapeutic dose of 20 ppm of nitric oxide is to be delivered to a patient, a gas including 20 ppm of a nitric oxide-releasing agent (e.g., NO₂) can be released from a gas bottle or a diffusion tube. The gas including 20 ppm of a nitric oxide-releasing agent can be passed through one or more cartridges to convert the 20 ppm of nitric oxide-releasing agent to 20 ppm of nitric oxide for delivery to the patient. However, in other delivery systems, the amount of nitric oxide-releasing agent in a gas can be greater than the amount of nitric oxide to be delivered to a patient. For example, a gas including 800 ppm of a nitric oxide-releasing agent can be released from a gas bottle or a diffusion tube. The gas including 800 ppm of a nitric oxide-releasing agent can be passed through one or more cartridges to convert the 800 ppm of nitric oxide-releasing agent to 800 ppm of nitric oxide. The gas including 800 ppm of nitric oxide can then be diluted in a gas including oxygen (e.g., air) to obtain a gas mixture with 20 ppm of nitric oxide for delivery to a patient. Traditionally, the mixing of a gas including nitric oxide with a gas including oxygen to dilute the concentration of nitric oxide has occurred in a line or tube of the delivery system. The mixing of a gas including nitric oxide with a gas including oxygen can cause problems because nitrogen dioxide can form. To avoid this problem, two approaches have been used. First, the mixing of the gases can be performed in a line or tube immediately prior to the patient interface, to minimize the time nitric oxide is exposed to oxygen, and consequently, reduce the nitrogen dioxide formation. Second, a cartridge can be placed at a position downstream of the point in the line or tubing where the mixing of the gases occurs, in order to convert any nitrogen dioxide formed back to nitric oxide.

While these approaches can minimize the nitrogen dioxide levels in a gas delivered to a patient, these approaches have some drawbacks. Significantly, both of these approaches mix a gas including nitric oxide with a gas including oxygen in a line or tubing of the system. One problem can be that lines and tubing in a gas delivery system can have a limited volume, which can constrain the level of mixing. Further, a gas in lines and tubing of a gas delivery system can experience variations in pressure and flow rates. Variations in pressure and flow rates can lead to an unequal distribution of the amount each gas in a mixture throughout a delivery system. Moreover, variations in pressure and flow rates can lead to variations in the amount of time nitric oxide is exposed to oxygen within a gas mixture. One notable example of this arises with the use of a ventilator, which pulses gas through a delivery system. Because of the variations in pressure, variations in flow rates and/or the limited volume of the lines or tubing where the gases are mixed, a mixture of the gases can be inconsistent, leading to variation in the amount of nitric oxide, nitrogen dioxide, nitric oxide-releasing agent and/or oxygen between any two points in a delivery system.

To address these problems, a mixing chamber can also be used to mix a first gas and a second gas. A first gas can include oxygen; more specifically, a first gas can be air. A second gas can include a nitric oxide-releasing agent and/or nitric oxide. A first gas and a second gas can be mixed within a chamber to form a gas mixture. The mixing can be an active mixing performed by a mixer within a chamber. For example, a mixer can be a moving support. The mixing within a chamber can also be a passive mixing, for example, the result of diffusion.

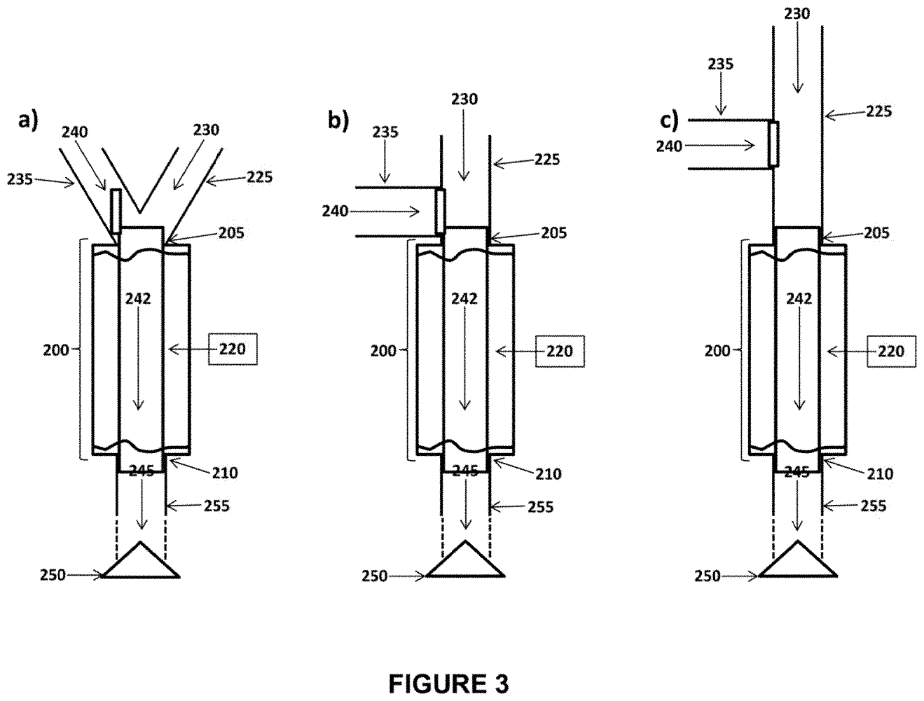

As shown in FIGS. 3a, 3b and 3c, a cartridge 200 can be coupled to a gas conduit 225. A first gas 230 including oxygen can be communicated through a gas conduit 225 to the cartridge 200. The communication of the first gas through the gas conduit can be continuous or it can be intermittent. For instance, communicating the first gas intermittently can include communicating the first gas through the gas conduit in one or more pulses. Intermittent communication of the first gas through gas conduit can be performed using a gas bag, a pump, a hand pump, an anesthesia machine or a ventilator.

A gas conduit can include a gas source. A gas source can include a gas bottle, a gas tank, a permeation cell or a diffusion tube. Nitric oxide delivery systems including a gas bottle, a gas tank a permeation cell or a diffusion tube are described, for example, in U.S. Pat. Nos. 7,560,076 and 7,618,594, each of which are incorporated by reference in its entirety. Alternatively, a gas source can include a reservoir and restrictor, as described in U.S. patent application Ser. Nos. 12/951,811, 13/017,768 and 13/094,535, each of which is incorporated by reference in its entirety. A gas source can include a pressure vessel, as described in U.S. patent application Ser. No. 13/492,154, which is incorporated by reference in its entirety. A gas conduit can also include one or more additional cartridges. Additional components including one or more sensors for detecting nitric oxide levels, one or more sensors for detecting nitrogen dioxide levels, one or more sensor for detecting oxygen levels, one or more humidifiers, valves, tubing or lines, a pressure regulator, flow regulator, a calibration system and/or filters can also be included in a gas conduit.

A second gas 240 can also be communicated to a chamber 200. A second gas can be supplied into a gas conduit, as shown in FIGS. 2b and 2c. Preferably, a second gas 240 can be supplied into a gas conduit 225 immediately prior to a chamber 200, as shown in FIG. 2b. A second gas 240 can be supplied into a gas conduit 225 via a second gas conduit 235, which can join or be coupled to the gas conduit 225. Once a second gas 240 is supplied into a gas conduit 225, both the first gas 230 and the second gas 240 can be communicated in the inlet 205 of a chamber 200 for mixing. Alternatively, a second gas 240 can be supplied at a chamber 200, as show in FIG. 2a. For example, a second gas 240 can be supplied directly into the inlet 205 of a receptacle 200.

Once a first gas 230 and a second gas 240 are within a chamber 200, a first gas 230 and a second gas 240 can mix to form a gas mixture 242 including oxygen and one or more of nitric oxide, a nitric oxide-releasing agent (which can be nitrogen dioxide) and nitrogen dioxide. The gas mixture 242 can contact a reducing agent, which can be on a support 220 within the chamber. The reducing agent can convert nitric oxide-releasing agent and/or nitrogen dioxide in the gas mixture to nitric oxide.

The gas mixture including nitric oxide 245 can then be delivered to a mammal, most preferably, a human patient. The concentration of nitric oxide in a gas mixture can be at least 0.01 ppm, at least 0.05 ppm, at least 0.1 ppm, at least 0.5 ppm, at least 1 ppm, at least 1.5 ppm, at least 2 ppm or at least 5 ppm. The concentration of nitric oxide in a gas mixture can be at most 100 ppm, at most 80 ppm, at most 60 ppm, at most 40 ppm, at most 25 ppm, at most 20 ppm, at most 10 ppm, at most 5 ppm or at most 2 ppm. Delivering the gas mixture including nitric oxide from the chamber 200 to the mammal can include passing the gas mixture through a delivery conduit. A delivery conduit 255 can be located between the chamber 200 and a patient interface 250. In some embodiments, a delivery conduit 255 can be coupled to the outlet 210 of a chamber 200 and/or coupled to the patient interface 250. As indicated by the dashed lines in FIGS. 2a, 2b and 2c, a delivery conduit can include additional components, for example, a humidifier or one or more additional cartridges.

Delivery of a gas mixture can include continuously providing the gas mixture to the mammal. When the delivery of the gas mixture includes continuously providing the gas mixture to the mammal, the volume of the receptacle or chamber can be greater than the volume of the delivery conduit. The larger volume of the receptacle can help to ensure that the gas mixture is being thoroughly mixed prior to delivery. Generally, more complete mixing can occur as the ratio of the volume of the receptacle to the volume of the delivery conduit increases. A preferable level of mixing can occur when the volume of the receptacle is at least twice the volume of the delivery conduit. The volume of the receptacle can also be at least 1.5 times, at least 3 times, at least 4 times or at least 5 times the volume of the delivery conduit.

When the volume of the receptacle is greater than the volume of the delivery conduit or the volume of gas mixture in the delivery conduit, the gas mixture may not go directly from the receptacle to the mammal, but instead, can be delayed in the receptacle or delivery conduit. It is this delay that can provide the time needed to mix the gas so that the NO concentration remains constant within a breath.

This delay can result in the storage of the gas mixture in the receptacle. The gas mixture can be stored in the receptacle for a predetermined period of time. The predetermined period of time can be at least 1 second, at least 2 seconds, at least 6 seconds, at least 10 seconds, at least 20 seconds, at least 30 seconds or at least 1 minute.

The mixing that occurs due to the delay of the gas mixture (i.e. storage of the gas mixture in a receptacle) can be so effective that the intra-breath variation can be identical to what could be achieved under ideal conditions when premixed gas was provided. This can be referred to as “perfect mixing.” For continuous delivery, this can mean that the concentration of nitric oxide in the gas mixture delivered to a mammal remains constant over a period of time (e.g. at least 1 min, at least 2 min, at least 5 min, at least 10 min or at least 30 min). For a concentration to remain constant, the concentration can remain with a range of at most ±10%, at most ±5%, or at most ±2% of a desired concentration for delivery.

Delivery of the gas mixture can include intermittently providing the gas mixture to the mammal. Intermittent delivery of a gas mixture can be the result of intermittent communication of a first or second gas into the system. Said another way, intermittent communication of a first or second gas through a gas conduit can result in an increased area of pressure, which can traverse into the receptacle causing intermittent communication of the gas mixture. Intermittent delivery can be performed using a gas bag, a pump, a hand pump, an anesthesia machine or a ventilator.

The intermittent delivery can include an on-period, when the gas mixture is delivered to a patient, and an off-period, when the gas mixture is not delivered to a patient. Intermittent delivery can include delivering one or more pules of the gas mixture.

An on-period or a pulse can last for a few seconds up to as long as several minutes. In one embodiment, an on-period or a pulse can last for 1, 5, 10, 15, 20, 25, 30, 35, 40, 45, 50, 55 or 60 seconds. In another embodiment, the on-period or a pulse can last for 1, 2, 3, 4 or 5 minutes. In a preferred embodiment, an on-period or a pulse can last for 0.5-10 seconds, most preferably 1-6 seconds.

Intermittent delivery can include a plurality of on-periods or pulses. For example, intermittent delivery can include at least 1, at least 2, at least 5, at least 10, at least 50, at least 100 or at least 1000 on-periods or pulses.

The timing and duration of each on-period or pulse of the gas mixture can be pre-determined. Said another way, the gas mixture can be delivered to a patient in a pre-determined delivery sequence of one or more on-periods or pulses. This can be achieved using an anesthesia machine or a ventilator, for example.

When the delivery of the gas mixture includes intermittently providing the gas mixture to the mammal, the volume of the receptacle can be greater than the volume of the gas mixture in a pulse or on-period. The larger volume of the receptacle can help to ensure that the gas mixture is being thoroughly mixed prior to delivery. Generally, more complete mixing can occur as the ratio of the volume of the receptacle to the volume of the gas mixture in a pulse or on-period delivered to a mammal increases. A preferable level of mixing can occur when the volume of the receptacle is at least twice the volume of the gas mixture in a pulse or on-period. The volume of the receptacle can also be at least 1.5 times, at least 3 times, at least 4 times or at least 5 times the volume of the gas mixture in a pulse or on-period.

When the volume of the receptacle is greater than the volume of the volume of the gas mixture in a pulse or on-period, the gas mixture may not go directly from the receptacle to the mammal, but instead, can be delayed in the receptacle or delivery conduit for one or more pulses or on-periods. It is this delay that can provide the time needed to mix the gas so that the NO concentration remains constant between delivered pulses or on-periods.

In addition to storage as a result of off-periods, the delay caused by the differing volumes can result in the storage of the gas mixture in the receptacle. The gas mixture can be stored in the receptacle for a predetermined period of time. The predetermined period of time can be during or between pulses or on-periods. The predetermined period of time can be at least 1 second, at least 2 seconds, at least 6 seconds, at least 10 seconds, at least 20 seconds, at least 30 seconds or at least 1 minute.

The mixing that occurs due to the delay of the gas mixture (i.e. storage of the gas mixture in a receptacle) can be so effective that the intra-breath variation can be identical to what could be achieved under ideal conditions when premixed gas was provided. Intermittent delivery an include providing the gas mixture for two or more pulses or on-periods. Using intermittent delivery, the concentration of nitric oxide in each pulse or on-period can vary by less than 10%, by less than 5%, or by less than 2%. In other words, the variation between the concentration of nitric oxide in a first pulse and the concentration of nitric oxide in a second pulse is less than 10% (or less than 5% or 2%) of the concentration of nitric oxide in the first pulse. In another embodiment, using intermittent delivery, the concentration of nitric oxide in each pulse or on-period can vary by less than 10 ppm, less than 5 ppm, less than 2 ppm or less than 1 ppm. Said another way, the difference between the concentration of nitric oxide in a first pulse and the concentration of nitric oxide in a second pulse is less than 10 ppm, less than 5 ppm, less than 2 ppm or less than 1 ppm.

### EXAMPLES

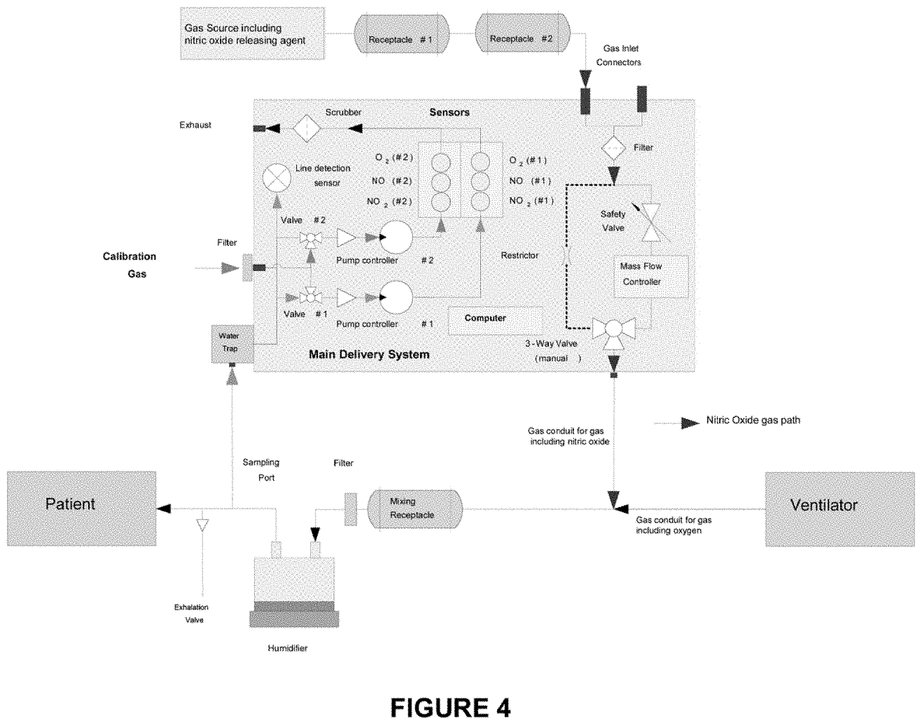

FIG. 4 shows the flow path schematics of an embodiment of a system where a receptacle is used for mixing gas. In this configuration, the gas source including a nitric oxide-releasing agent can be NO₂ in air, for example a bottle of 800 ppm NO₂ in air. Alternatively, the gas source can also be from a liquid source. If a liquid source is used, then the concentration of the source can be variable. In some instances, the concentration of NO₂ can be from about 1000 ppm down to about 50 ppm. The concentration of NO₂ from a liquid source can be controlled by controlling the temperature of the source.

The embodiment shown in FIG. 3 has demonstrated the ability to supply a constant concentration of NO for the duration of the inspired breath. The functions of a receptacle, shown as a mixing receptacle in FIG. 3, can include:

1) To convert any NO₂ that may have formed in the line into NO.

2) To provide adequate mixing of NO in the patient circuit prior to inhalation.

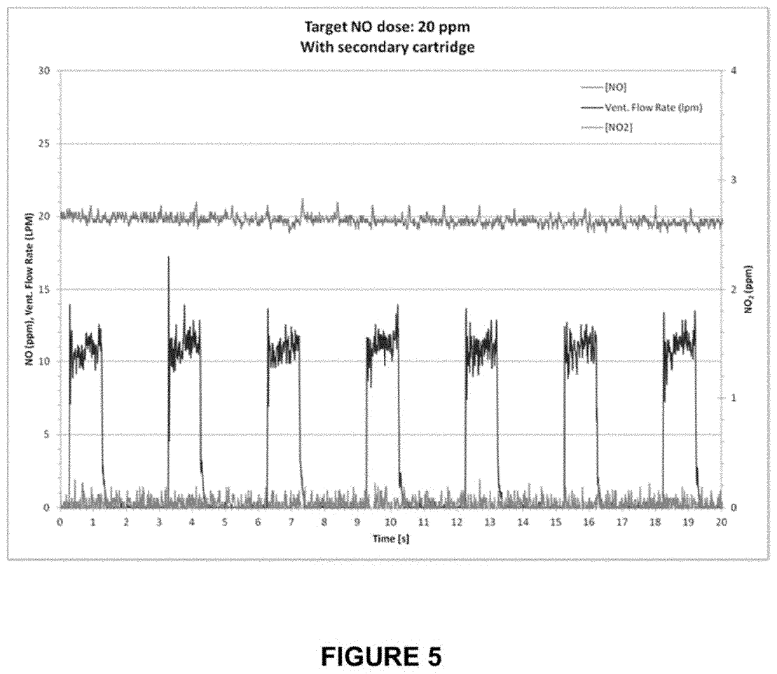

FIG. 5 shows a typical response of a system as embodied in FIG. 3 configured to deliver 20 ppm of NO. The NO₂ values (bottom) are shown (right hand axis). These measurements were obtained using the electrochemical gas analyzers that are part of the system. It is to be noted that the NO₂ levels can be essentially zero when the NO level is at 20 ppm. As shown by the middle plot, the ventilator flow rate is shown (left hand axis). To focus on the worst case scenario, the bias flow of the ventilator was set to zero.

The system was delivering 20 ppm of NO in 21% oxygen using an infant ventilator (Bio-Med Devices CV2+) with the ventilator settings shown in Table 1. The slower breathing rate was used as the worst case for NO mixing, because of the longer pause during exhalation.

TABLE 1
Ventilator Settings
Ventilator Settings

| Mode | Pressure Control |
|---|---|
| Rate (BPM) | 40 |
| Inspiratory Time INSP (sec) | 0.50 |
| Flow (LPM) | 6.0 |
| I:E Ratio | 1:2.0 |

The NO measurements were within product specifications (±20%). The conversion of NO₂ to NO in the receptacle overcomes the formation of NO₂ that is caused by the delay due to mixing.

As discussed above, the mixing can occur if the volume of the receptacle exceeds the ventilator pulse volume. For example, a 6000 ml/min and 40 breaths per minute the volume of the pulse is 150 ml. Good mixing can occur as long as the volume of the mixing chamber is greater than twice this volume.

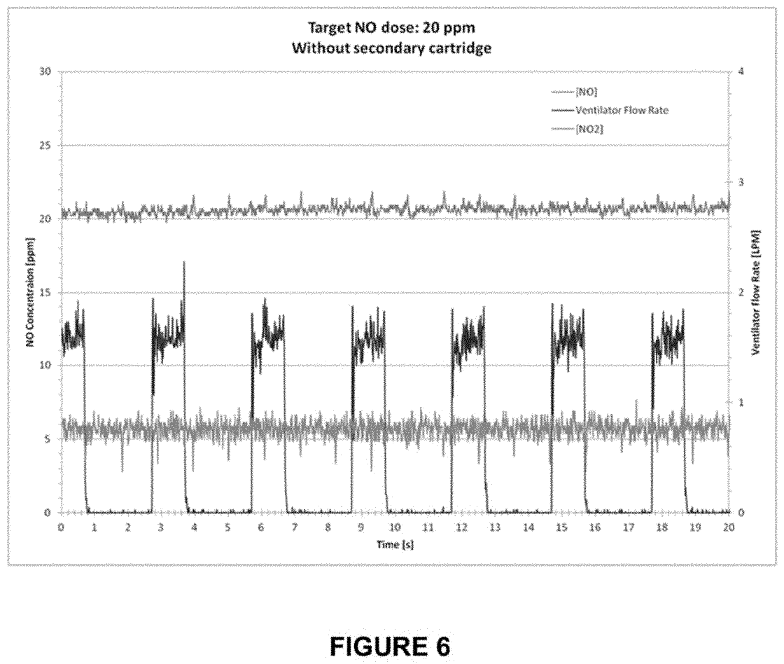

On the other hand, FIG. 6 shows the same response but without the receptacle, shown as the mixing receptacle in FIG. 3, in line with the patient. The NO₂ levels read around 0.6 ppm, which would be unacceptable for a neonate. The receptacle converts all of the NO₂ that was formed back into NO. These two figures clearly demonstrate the effect of a receptacle for converting NO₂ into NO, namely the receptacle reduced the NO₂ level as measured at the patient from 0.6 to 0 ppm.

The mixing performance of the receptacle was assessed using a high speed chemiluminescence detector with a 90% rise time of 250 msec. A very high speed NO detector was needed to catch the intra-breath variability of nitric oxide.

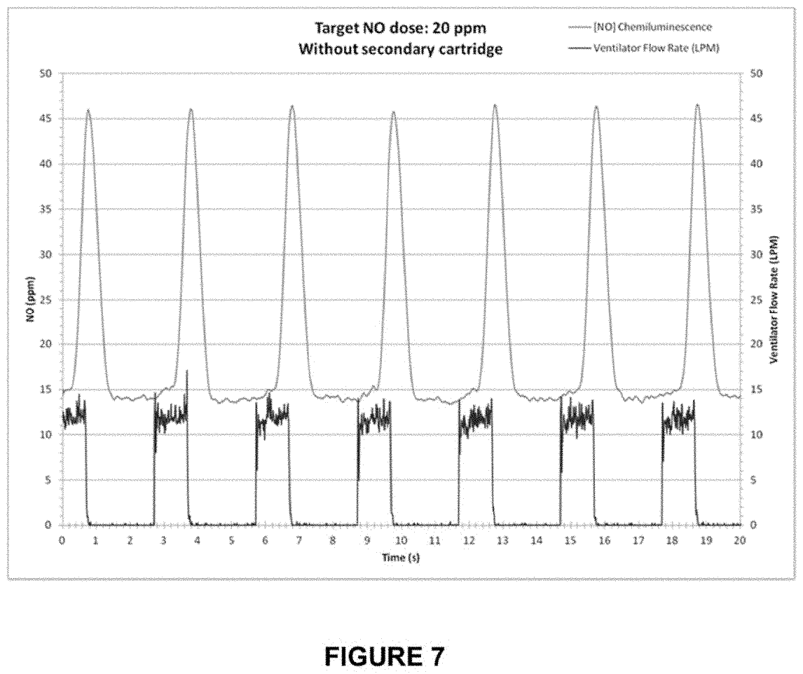

FIG. 7 shows the response of the system without the receptacle for mixing the gases (no mixing function). This chart shows the high speed version of the NO waveform presented in FIG. 5. The bottom line shows the flow rate of the ventilator. As can be seen, the absence of the receptacle introduced spikes of 30 ppm of nitric oxide (top) during the inspiratory time. Intra-breath variability of this magnitude is unacceptable.

Previous technology partially solved this problem by tracking the rapid intra-breath flow changes in the ventilator circuit and uses the electronic signal from the flow sensor to synchronize the valve that introduces the NO into the circuit. This is a difficult and complex electronic solution that requires high speed sensors and very fast computer algorithms operating in real time. Because it is so difficult to execute, the FDA (in their Guidance document) allows the NO to vary from 0 to 150% of the mean, if the total duration of these transient concentrations did not exceed 10% of the volumetric duration of the breath.

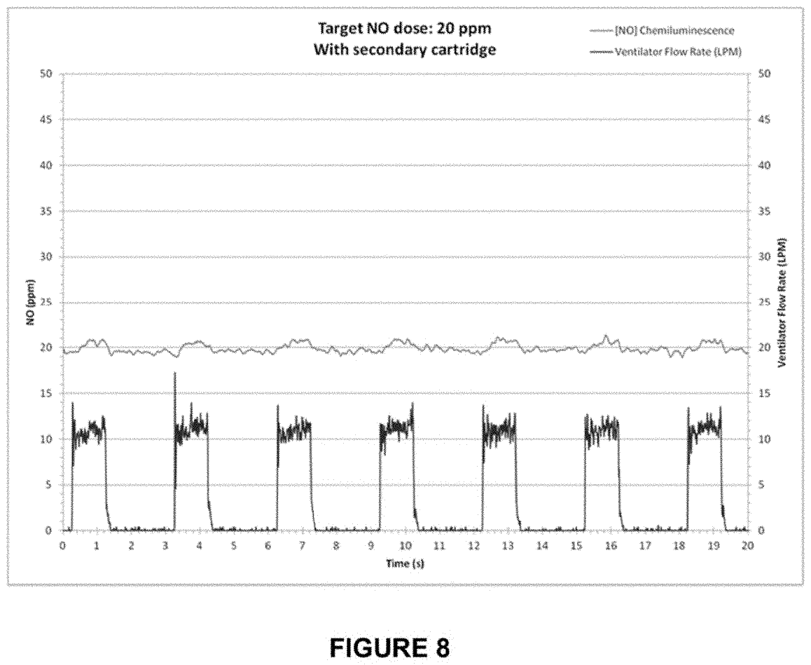

FIG. 8 shows the high speed NO version of FIG. 5 including a receptacle. The high speed detector was able to detect intra-breath variations as low as 1 ppm for the same ventilator settings used in FIG. 7. (In FIG. 5, the pulsations are not shown on the NO reading since the time response of the electro-chemical cell and associated electronics was significantly greater than the time between breaths.) The only difference was the addition of the receptacle which provides the mixing function.

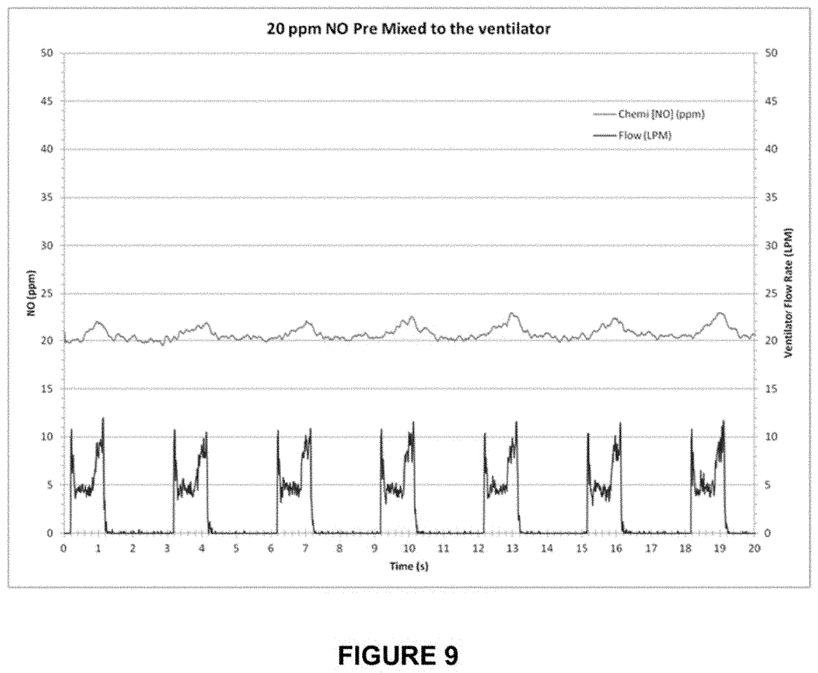

Ideal mixing can happen when the NO gas is premixed and delivered directly using the ventilator. This perfect mixing condition can provide a baseline in order to validate chemiluminescence measurements under pulsing conditions. A blender was used to premix 800 ppm of NO with air to generate a 20 ppm gas to be delivered using a ventilator only. Chemiluminescence was used to measure the NO delivered to the artificial lung. FIG. 9 shows the results. From the peaks in the NO plot (top), it is evident that the chemiluminescence device was affected by the pulsing nature of the flow (bottom). The NO measurements were almost flat but some variations were still present.

FIG. 10 shows the same experiment but the system includes a receptacle within the breathing circuit. The small amplitude oscillations were present in the NO measurements (top). From these simple experiments, it was concluded that the pulsing flow from the ventilator can provide a perfectly flat NO response using the chemiluminescence device. Furthermore, these oscillations may be due to the pressure changes in the breathing circuit since they were synchronized with the ventilator flow rate measurements (bottom). The intra breath variation that was achieved by mixing in the cartridge was indistinguishable from ideal and what can be achieved using premixed gases. In addition, the NO₂ impurity level is reduced to almost 0.0 ppm.

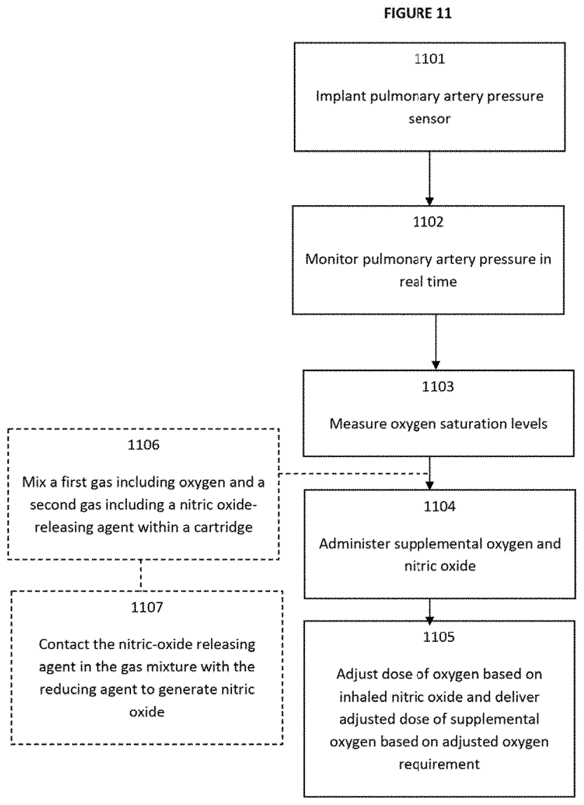

FIG. 11 shows an embodiment of the invention. The method includes implanting a pulmonary artery pressure sensor (1101), monitoring pulmonary artery pressure in real time (1102), measuring oxygen levels in a patient (1103), administer supplemental oxygen and nitric oxide (1104), and adjusting dose of oxygen based on inhaled nitric oxide and deliver adjusted dose of supplemental oxygen based on adjusted oxygen requirement (1105). In certain embodiments, the method can optionally include mixing a first gas including oxygen gas and a second gas including a nitric-oxide releasing agent within a cartridge (1106) and then contacting the nitric oxide-releasing agent with the reducing agent to generate nitric oxide (1107). The method can further include determining a first oxygen requirement based on a patient's condition or disease state, for example. Upon determining an oxygen requirement, a clinician such as a physician or other professional or person operating in a health care capacity, can then adjust the dose of oxygen in real time to a second dose based on the inhaled nitric oxide. The clinician can determine a reduced oxygen requirement in view of the inhaled nitric oxide, either before or after the dose of oxygen is adjusted to a second dose or titrated until a target level of oxygen is reached. After a reduced oxygen requirement is determined or adjusted, a clinician can deliver a dose of supplemental oxygen based on the reduced oxygen requirement and the gas mixture including nitric oxide.

Constant NO injection into the breathing circuit can be a simple and viable technique as long as a receptacle is both a mixer with sufficient volume and can remove NO₂ from the circuit or can convert the NO₂ back into NO.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

1. A method of modulating oxygen saturation levels, comprising:
measuring oxygen saturation levels in a patient;
administering inhaled nitric oxide;
adjusting the dose of oxygen in real time to a second dose based on the inhaled nitric oxide;
determining a first oxygen requirement to address an oxygen deficiency;
determining a reduced oxygen requirement based on the generated nitric oxide; and
delivering a dose of supplemental oxygen based on the reduced oxygen requirement and the gas mixture including nitric oxide from the receptacle to the patient.

2. The method of claim 1 further comprising
mixing a first gas including oxygen and a second gas including a nitric oxide-releasing agent within a receptacle to form a gas mixture, wherein the receptacle includes an inlet, an outlet and a reducing agent; and
contacting the nitric oxide-releasing agent in the gas mixture with the reducing agent to generate nitric oxide

3. The method of claim 1 wherein adjusting the dose includes titrating the dose of oxygen in real time.

4. A method of modulating oxygen saturation levels, comprising:
measuring oxygen saturation levels in a patient;
determining a first dose of oxygen to address an oxygen deficiency;
mixing a first gas including oxygen and a second gas including a nitric oxide;
determining a second dose of oxygen based on an amount of nitric oxide to be co-administered with the oxygen, wherein the second dose is lower than the first dose; and
delivering the gas mixture including nitric oxide from the receptacle to the patient.

5. The method of claim 1, wherein the method includes an incremental reduction of pO2.

6.-12. (canceled)

13. The method of claim 1, wherein the concentration of nitric oxide in the gas mixture delivered is at least 0.01 ppm and at most 2 ppm.

14. The method of claim 1, wherein hydrogen is added in the following combinations: (H+O2) or (H+NO) or (H+NO+O2).

15. (canceled)

16. The method of claim 1, further comprising delivering hydrogen, the hydrogen acts to eliminate peroxynitrite, thereby reducing adverse effects of nitric oxide.

17.-20. (canceled)

21. The method of claim 2, wherein delivering the gas mixture including nitric oxide from the receptacle to the mammal includes pulsing the gas mixture.

22. The method of claim 21, wherein pulsing includes providing the gas mixture for one or more pulses of 1 to 6 seconds.

23.-28. (canceled)

29. The method of claim 2, comprising communicating the first gas through a gas conduit to the receptacle and supplying the second gas into the gas conduit immediately prior to the receptacle.

30.-34. (canceled)

35. The method of claim 1, wherein the nitric oxide is administered to adults.

36. The method of claim 1, wherein the nitric oxide is provided through a cartridge having a length, width, and thickness, an outer surface, and an inner surface, and can be substantially cylindrical in shape.

37. The method of claim 36, wherein the thickness between the inner and outer surface is constant, thereby providing a uniform exposure to the reducing agents.

38. (canceled)

39. A method of modulating oxygen saturation levels, comprising:
implanting a pulmonary artery pressure sensor;
monitoring pulmonary artery pressure in real time;
measuring oxygen saturation levels in a patient;
administering inhaled nitric oxide;
adjusting the dose of oxygen in real time to a second dose based on the inhaled nitric oxide;
determining a first oxygen requirement to address an oxygen deficiency;
determining a reduced oxygen requirement based on the generated nitric oxide; and
delivering a dose of supplemental oxygen based on the reduced oxygen requirement and the gas mixture including nitric oxide from the receptacle to the patient.

40. The method of claim 39, wherein the pulmonary artery pressure sensor is configured to monitor the right heart.

41. The method of claim 39, wherein the pulmonary artery pressure sensor is configured to monitor the left heart.

42. The method of claim 39, wherein the pulmonary artery pressure sensor is a wireless device.
